(19) United States
(12) Patent Application Publication
(10) Pub. No.: US 2026/0311801 A1
(43) Pub. Date: Oct. 8, 2026

(54) SYSTEM AND METHOD FOR DETERMINING CANDIDATE RNA BINDERS

(71) Applicant: Ladder Therapeutics Inc, Portland, OR (US)

(72) Inventors: Timothy Allen, Portland, OR (US); Rabia Khan, Portland, OR (US)

(73) Assignee: Ladder Therapeutics Inc, Portland, OR (US)

(21) Appl. No.: 19/638,950

(22) Filed: Apr. 3, 2026

Related U.S. Application Data
(60) Provisional application No. 63/783,127, filed on Apr. 3, 2025.

(51) Int. Cl. G16B 15/30; G16B 15/10; G16B 35/20; G16B 40/20

(52) U.S. Cl. CPC G16B 15/30; G16B 15/10; G16B 35/20; G16B 40/20

(57) ABSTRACT

The method can include determining RNA binding data; identifying a molecule and/or fragment of interest; determining candidate molecules based on the molecule and/or fragment of interest; and optionally evaluating the candidate molecules. The system can include a database of RNA binding data, a set of representation and/or embedding models, and a set of search and/or molecule generation models. The system and method can function to identify and/or prioritize candidate molecules (e.g., potential hits).

### CROSS-REFERENCE TO RELATED APPLICATIONS

This application claims the benefit of U.S. Provisional Application No. 63/783,127 filed 3 Apr. 2025, which is incorporated in its entirety by this reference.

### TECHNICAL FIELD

This invention relates generally to the cheminformatics field, and more specifically to a new and useful system and method for determining candidate RNA binders in the cheminformatics field.

### BRIEF DESCRIPTION OF THE FIGURES

FIG. 1 is a schematic representation of a variant of the method.

FIG. 2 is a schematic representation of a variant of the system.

FIG. 3 is a schematic representation of an example of determining candidate molecules.

FIG. 4 is a schematic representation of an example of determining candidate molecules and evaluating candidate molecules

FIG. 5A depicts a specific example of building an enriched chemical library.

FIG. 5B depicts a specific example of a splicing targeting library.

FIGS. 6A-6C depicts examples of using representation(s) of one or more molecules of interest to determine a set of candidate molecules.

FIGS. 7A-7C depicts examples of generating new candidate molecules and evaluating the new candidate molecules.

FIGS. 8A-8C depicts specific examples of generating new candidate molecules.

FIG. 9 depicts an illustrative example of RNA binding data. Left experimentally collected RNA Binding data from a single screen, compounds are shown on the y-axis and RNA targets on the x-axis. A binding interaction is noted by a teal box, black indicates no interaction. The bottom section of this diagram is all compounds with no binding to any targets. Right-the non-binders are removed focusing on binders. RNA targets have been ordered into structural classes on the x-axis as labelled. Compounds have been ordered to draw out structure selectivity in the lower section of this diagram (i.e. compounds that bind to at least one G4 target but no targets of other classes).

FIG. 10 is a schematic representation of a variant of determining candidate molecules.

FIG. 11 is a schematic representation of a variant of predicting molecule or fragment thereof binding to an RNA motif.

### DETAILED DESCRIPTION

The following description of the embodiments of the invention is not intended to limit the invention to these embodiments, but rather to enable any person skilled in the art to make and use this invention.

### 1. Overview

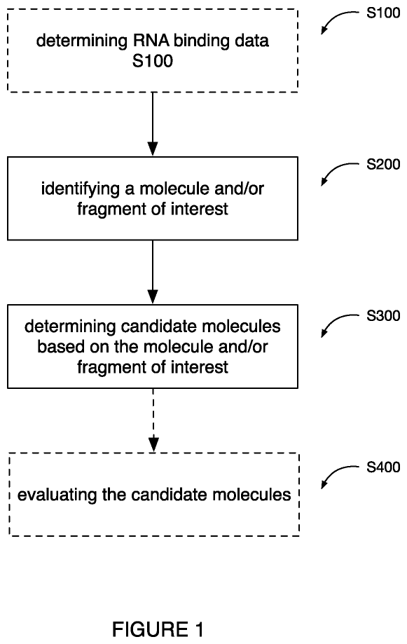

As shown in FIG. 1, the method can include: optionally determining RNA binding data S100; identifying a molecule and/or fragment of interest S200; determining candidate molecules based on the molecule and/or fragment of interest S300, and optionally evaluating the candidate molecules S400. However, the method can additionally or alternatively include any other suitable steps.

In variants, the method can function to identify and/or prioritize candidate molecules (e.g., potential hits). While typically used to generate and select candidate hits for RNA binding, the method is not limited to these applications. Additional or alternative applications of variations of the system and/or method can include generating and selecting candidate hits for antibacterial properties, antiviral properties, antifungal properties, prion disease targets, ability to pass through the blood-brain barrier, anti-viroid properties, antiprotozoan properties, autoimmune targets, genetic disorder targets, cancer targets, and/or other suitable targets (using appropriate assays to evaluate the candidate molecules, train the models, etc. for these applications).

### 2. Examples

In an example, the method can include: performing a hit expansion by using a molecule and/or fragment of interest (a hit) to search through known molecules for candidate molecules and/or using the molecule and/or fragment of interest to generate new candidate molecules. In a first example, a search model can: determine a structural, physiochemical, and/or machine learning representation (e.g., fingerprint) of the molecule and/or fragment of interest, and select the candidate molecules based on the molecule and/or fragment of interest representation. In a specific example, the candidate molecules can be selected by calculating a similarity metric for each candidate molecule (e.g., based on the molecule and/or fragment of interest representation and the respective candidate molecule representation), and comparing each calculated similarity metric to a threshold value (e.g., a learned threshold value, a learned number of nearest neighbours, etc.). In a second example, a generation model can generate new chemicals (e.g., as SMILES strings, chemical structures, etc.) for candidate molecules based on a SMILES string for the molecule and/or fragment of interest. The generation model can optionally include an ensemble of models (e.g., combinations of two or more of an LLM, MMP, graph, transformer, etc.), executed in parallel. The model(s) (e.g., a latent space therein) can optionally be tuned using RNA binding data and/or other functional data (e.g., measured using an assay).

Downstream processes using the candidate molecules can include: validating the candidate molecules as confirmed hits using assays, evaluating the candidate molecules (e.g., for drug discovery), building an enriched chemical library, and/or any other suitable uses. In an example, evaluating the candidate molecules can include ranking the candidate molecules using a multi-parametric optimization with respect to multiple parameters and objectives (e.g., using in silico docking models, ligand methods, structure-based methods, pharmacophore methods, etc.).

### 3. Technical Advantages

Variants of the technology can confer one or more advantages over conventional technologies.

First, variants of the technology can increase the hit rates of primary screens by identifying and/or generating a set of candidate molecules with an increased likelihood of being a hit. For example, given a query molecule (e.g., a known hit), the set of candidate molecules can be determined using a representation (e.g., a structural, physiochemical, and/or a machine learning representation) for the query compound. This representation can be trained using datasets of known RNA hits (e.g., binders or other materials with desirable RNA interactions), which can bias the molecular search space toward molecules more likely to be hits. In an example, the models can be trained using datasets relevant to specific RNA structures (e.g., multiway junction, stem loop, G-quadruplex, hairpin loops, external loops, internal loops, multihelix, junction loops, bulge loops, multiple branch loops, pseudoknots, etc.) and/or functional targets (e.g., translation; splicing modulators; existing antibiotics such as macrolides, sofosbuvir, streptomycin, doxycycline, erythromycin, linezoid, etc.; existing medications such as Risdiplam, quarfloxin, CX-5461, branaplam, etc.; etc.). In variants, this targeted approach can lead to more efficient discovery of RNA-binding compounds compared to conventional methods that may rely on protein-targeting compound databases or other, non-RNA binding datasets. Furthermore, the use of the trained representation can enable more molecules to be identified relative to conventional methods, as well as more varied molecules to be identified. In a specific example, candidate molecules that are close to the query molecule within the representation latent space that would not otherwise be identified as similar using conventional search methodologies. Moreover, variations of these methods can be used to scaffold hop and/or otherwise move outside of classical chemical space for an input hit. For example, novel antibiotics can be identified that would have activity against the known target of bacterial ribosomal RNA and are outside the known resistance mechanisms. As another example, the method can generate analogues of existing functional targets, where the analogues are outside of the chemical space known to engineer selectivity of an analogue.

Second, variants of the technology can facilitate the construction of specialized chemical libraries enriched for specific structure and/or function targets. The system can generate collections of compounds predicted to interact with specific RNA structures (e.g., multiway junction, stem loop, G-quadruplex, hairpin loops, external loops, internal loops, multihelix, junction loops, bulge loops, multiple branch loops, pseudoknots, etc.) and/or modulate specific cellular processes (e.g., translation, splicing, etc.). By focusing on RNA-specific chemical space, these libraries can provide more relevant starting points for RNA-targeted drug discovery compared to conventional libraries that may contain predominantly protein-targeting compounds. For example, the technology can generate focused libraries of small molecules predicted to bind specific RNA structure elements, which can accelerate the identification of RNA-targeting small molecules.

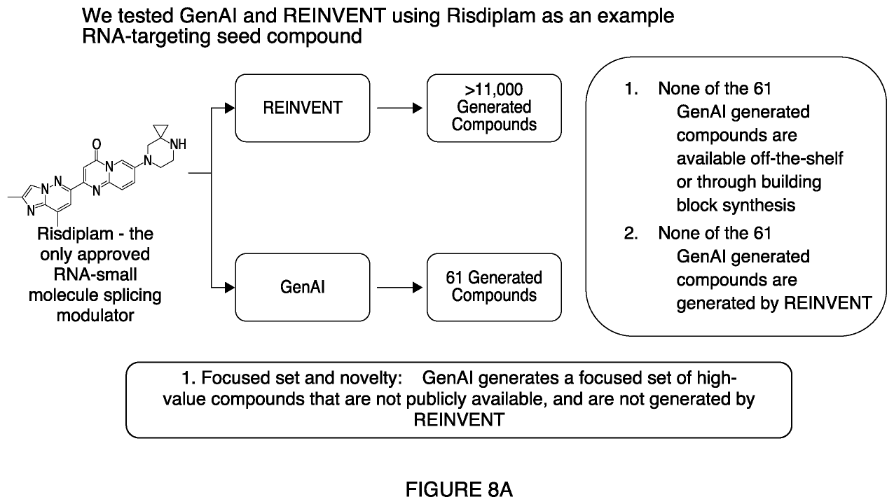

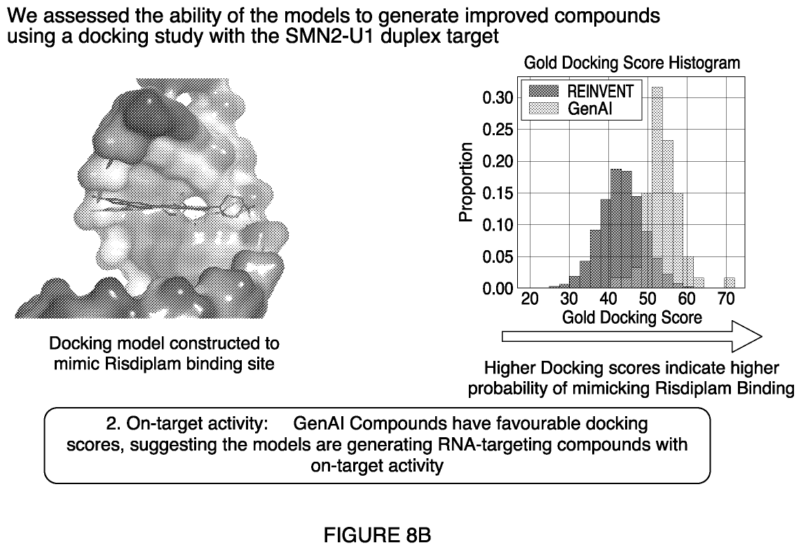

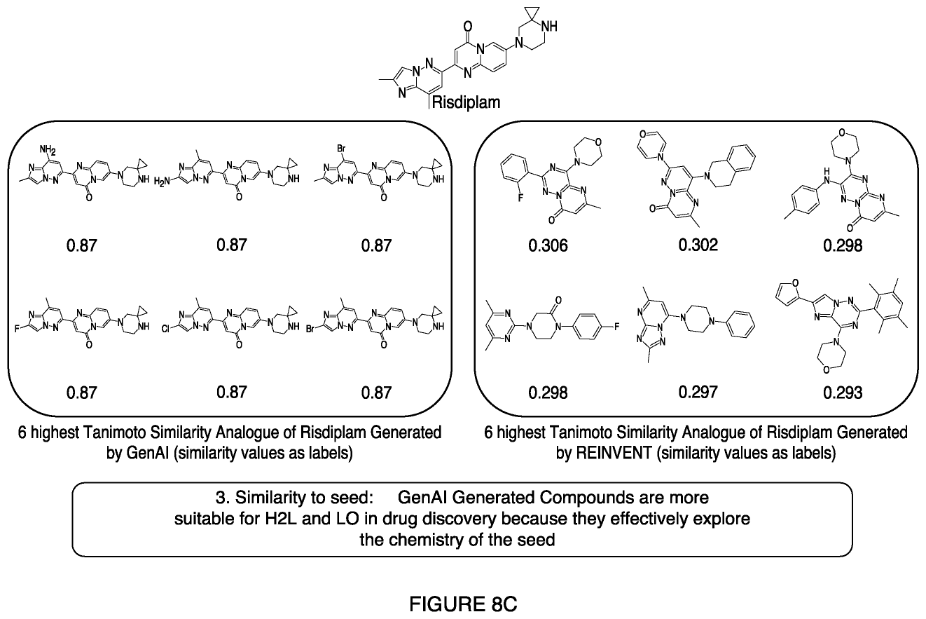

Third, variants of the technology can identify (e.g., generate) chemicals matter not designed by other generative chemistry algorithms or human chemists (as shown for example in FIG. 8A). In tests, these identified compounds have been found to target RNA (as shown for example in FIG. 8B) and thus variants of the method have been shown to identify molecules outside of an expected parameter space with desirable properties.

Fourth, variants of the method can enable ranking of multiple priorities contemporaneously, which can result in fewer iterations to identify candidate molecules (e.g., potential hits) and/or with better predictive outcomes. For instance, examples of the method can generate novel chemicals as candidate hits, rank the candidate hits on a set of parameters, and screen the candidate hits based on the ranking to reduce timeline for discovery of new drugs. In these examples, the ranking and/or screening can be optimized across multidimensional space (e.g., using multiple parameters) resulting in drastically reduced timelines (as compared to humans who have a finite set of chemical iterations to candidate hits). As a non-limiting example, the candidate hits can be ranked based on RNA-targeting predictions of activity and/or specific bespoke models for the chemical series of interest.

However, further advantages can be provided by the system and method disclosed herein.

### 4. System

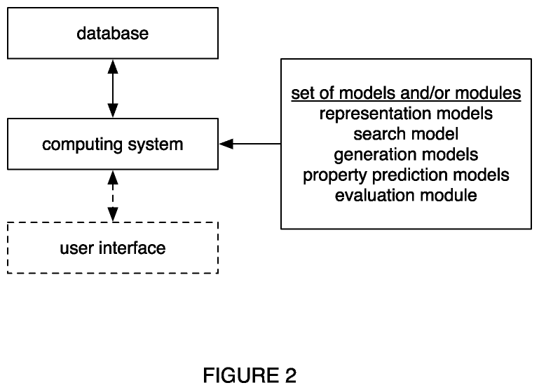

All or portions of the method can be performed by a system (e.g., platform) including one or more of: a computing system, a database 100 (e.g., a system database, a third-party database, etc.), a user interface 200 (e.g., mobile application, web application, desktop application, API, database, etc.), a set of models, and/or any other suitable system components. An example is shown in FIG. 2.

The computing system can include one or more: CPUs, GPUs, TPUs, custom FPGA/ASICS, microprocessors, servers, cloud computing, and/or any other suitable components. The computing system can be local, remote (e.g., cloud computing server, etc.), distributed, and/or otherwise arranged relative to any other system or module. The computing system can optionally interface with one or more databases and/or the user interface.

The database can include data on one or more of: molecule-RNA interaction data, binding data specific to one or more RNA structure classes (e.g., multiway junction, stem loop, G-quadruplex, hairpin loops, external loops, internal loops, multihelix, junction loops, bulge loops, multiple branch loops, pseudoknots, etc.), binding data specific to one or more functions (e.g., splicing, translation, RNA binding protein-RNA binding, RNA-RNA binding, IncRNA-RBP binding, etc.), small molecules (e.g., SMILES strings for a set of known small molecules), and/or any other data. In an example, the database can include experimental assays (e.g., performed using ALIS, ASMS, etc.) designed to evaluate molecule-RNA interactions. The database can optionally include or interface with data in one or more third-party databases (e.g., Enamine, OTAVA, etc.).

The user interface can receive one or more inputs (e.g., from a user), display one or more outputs (e.g., model outputs), display any other parameters, and/or otherwise function. In an example, the user interface can receive one or more: molecules of interest (e.g., query compounds), objectives (e.g., for molecule evaluation), and/or any other suitable user inputs.

The system can include or use one or more models (e.g., representation models, search models, generation models, predictive models, etc.). In variants, the models and/or modules can include a representation model 300, a search model 400, a set of molecule generation models 500, optionally a set of property prediction models 600, an evaluation module 700, and/or any other suitable models and/or modules.

The models can use classical or traditional approaches, machine learning approaches, and/or be otherwise configured. The models can use or include regression (e.g., linear regression, non-linear regression, logistic regression, etc.), decision tree, clustering, association rules, dimensionality reduction (e.g., PCA, t-SNE, LDA, etc.), language processing techniques (e.g., LSA), neural networks (e.g., GNN, CNN, DNN, CAN, LSTM, RNN, FNN, encoders, decoders, deep learning models, transformers, etc.), ensemble methods, optimization methods (e.g., Bayesian optimization, multiparametric optimization methods, etc.), classification, rules, heuristics, equations (e.g., weighted equations, etc.), selection (e.g., from a library), lookups, regularization methods (e.g., ridge regression), Bayesian methods (e.g., Naïve Bayes, Markov, etc.), instance-based methods (e.g., nearest neighbor), kernel methods, support vectors (e.g., SVM, SVC, etc.), statistical methods (e.g., probability), comparison methods (e.g., matching, distance metrics, thresholds, etc.), deterministic methods, genetic programs, foundation models, large language models (e.g., Bidirectional Encoder Representations from Transformers (BERT)), matched molecular pairs (MMP), and/or any other suitable model. In preferred variants, an ensemble of models is used (e.g., two or more of cheminformatics models, ML models, scaffold hopping, MMPs, etc.).

Models can be trained (e.g., tuned), learned, fit, predetermined, and/or can be otherwise determined. The models can be trained or learned using: supervised learning, unsupervised learning, self-supervised learning, semi-supervised learning (e.g., positive-unlabeled learning), reinforcement learning, transfer learning, Bayesian optimization, fitting, interpolation and/or approximation (e.g., using gaussian processes), backpropagation, and/or otherwise generated. The models can be learned or trained on: labeled data (e.g., data labeled with the target label), unlabeled data, positive training sets (e.g., a set of data with true positive labels), negative training sets (e.g., a set of data with true negative labels), and/or any other suitable set of data.

In variants, the representation model 300 functions to determine representations of molecules (e.g., molecules of interest, fragments of molecules, known molecules, candidate molecules, etc.) and/or RNA targets (e.g., RNA sequence of interest, RNA structure class of interest, etc.). In variants, the representation can perform a transformation on a hit and/or RNA data to represent the data in a different feature space. The representation model 300 preferably receives as input chemical strings (e.g., Simplified Molecular Input Line Entry System (SMILES), SMILES arbitrary target specification (SMARTS), international chemical identifier (InChI), etc.) and RNA sequences, but can additionally and/or alternatively receive molecular graphs, RNA secondary structures (e.g., dot-bracket notation), RNA tertiary structure representations, nucleotide k-mer representations, chemical fingerprints, 3D structures of molecules, molecular descriptors, molecule names, chemical moieties (e.g., for molecular fragments), and/or any other representations of a molecule or RNA target. The representation model 300 preferably generates, as output, machine learning representations (e.g., embeddings, latent space embeddings, feature vectors, feature maps, language-based embeddings, graph-based embeddings, etc.), but can additionally and/or alternatively generate structural representations (e.g., molecular graphs, adjacency matrices, distance matrices, contact maps, 3D representations, etc.), binary fingerprints, continuous fingerprints, learned molecular descriptors, and/or any other suitable representation. However, the representation model 300 can produce any output representation based on any input data.

The representation model can include neural networks (e.g., MLP, CNN, GNN, DNN, RNN etc.), transformers, probabilistic models, statistical models, linear maps, pre-trained models, rules, heuristics, and/or any other suitable model. In variants, the representation model 300 can be an encoding arm and/or the embedding layers of an autoencoder or any other model. For example, embedding layers of a model trained to generate molecules (e.g., molecule generation model 500), predict function, predict structure, and/or perform any other function, can be extracted to determine the representation model 300. In these variants, the representation model 300 can be trained to determine embeddings in a latent space that can encode structural relationships, functional attributes, chemical properties (e.g., binding affinity, functional activity, etc.), and/or other features relevant to downstream prediction tasks, optimization tasks, generation tasks, and/or any other tasks. In variants, the representation model can be the embedding and/or encoding layers of the search model 400, the molecule generation model(s) 500, the property prediction model(s) 600, and/or any other suitable prediction model. In these variants, the representation model and the prediction models can be trained end-to-end. In other variants, the representation models can be trained separately from the search model 400, the molecule generation model(s) 500, the property prediction model(s) 600, and/or any other prediction models.

However, the representation model 300 can be otherwise configured.

In variants, the search model 400 functions to determine a set of candidate molecules from a database of known molecules based on a molecule of interest (e.g., hit). In specific variants, the search model can perform hit expansion by finding molecules and/or molecular fragments similar to a hit. This can allow a user to find molecules that may bind to a specific RNA motif based on a known binding molecule. The search model 400 can receive, as input, a SMILE string, an embedding (e.g., determined from the representation model 300), and/or any other suitable data representation (e.g., of the hit molecule or fragment). The search model 400 can produce, as output, a list of molecules (e.g., SMILE strings, molecular formulas, molecular graphs, etc.) from a known database (e.g., based on a similarity score, based on a search, etc.). In variants, the search model can determine representations (e.g., using the representation model 300) for the hit molecule and the known molecules, such that they are in the same space (e.g., feature space, latent space, property space, etc.). The search model can determine candidate molecules within the shared space. However, the search model can otherwise function.

In variants, the search model 400 can perform a search (e.g., within a latent and/or feature space, a feature and/or latent space determined by the representation model S400, in property space, based on molecule properties, etc.). Performing a search in latent and/or feature space can include performing nearest neighbor search, k-nearest neighbor search, radius-based neighbor search, similarity threshold search, embedding space traversal, optimization-guided search, and/or any other search algorithm. In variants, the search model 400 can leverage clustering algorithms (e.g., to determine clusters and/or groups of similar molecules, etc.). Examples of clustering algorithms can include k-means clustering, hierarchical clustering, density-based spatial clustering of applications with noise (DBSCAN), Gaussian mixture models, spectral clustering, affinity propagation, mean-shift clustering, self-organizing maps, agglomerative clustering, graph-based clustering, community detection algorithms, and/or any other clustering method configured to group similar molecules or representations. In variants, performing search and/or clustering within a learned embedding space can determine candidate molecules based on encoded information and/or relationships not easily distinguishable by semantic data. For example, a feature space generated by a model trained to predict RNA binding probabilities can encode patterns and relationships relevant to RNA binding, such that performing a similarity search within this feature space more accurately identifies additional molecules likely to bind the RNA target. However, in variants, the search model can perform search and/or clustering in property space, descriptor space, and/or any other suitable space.

In variants, the search model can compute a similarity metric between the molecule of interest and/or fragments thereof and the known molecules or fragments thereof. In a first variant, the similarity metric can be a predefined computation and/or geometric measurement (e.g., based on the representation determined by the representation model 300). For example, the similarity metric can include: a distance within the latent space, a dot product, a ratio, Tanimoto coefficient, Jaccard similarity, Dice index, Cosine coefficient, Soergel distance, Euclidean distance, Manhattan distance, Mahalanobis distance, Pearson correlation, and/or any other comparison between representations. In an example, the candidate molecules can be the molecules within a threshold distance of the molecule of interest in the latent space (e.g., within a threshold radius around the molecule of interest).

In a second variant, the similarity metric can be determined using a machine learning model trained to output a similarity score between molecules, where the similarity score can be based on the SMILES strings, molecule representations (e.g., determined by a representation model 300), learned embeddings, learned transformations, or other representation. In an example, the candidate molecules can be the molecules above a threshold similarity score.

In variants, in which the search model includes a machine learning model that predicts a similarity score, the machine learning model can be trained and/or tuned based on experimental binding measurements, computationally predicted binding affinities, molecular property data, structural or conformational data, known ligand-RNA interaction data, literature-derived annotations, high-throughput screening results, expert-curated datasets, simulated docking scores, and/or any other data suitable for supervising or refining the similarity predictions. For example, training the machine learning model can include using data labelled based on functional information, molecular information, structural information, and/or any other information. In these variants, the machine learning model can be trained to determine similarity scores based on function, chemistry, structure, and/or any other properties.

In variants, the search model can be utilized with a plurality of representations (e.g., determined from the representation models 300). For example, the search model can be used to determine candidate molecules within a first embedding space and candidate molecules within a second embedding space. The two lists of candidate molecules can be aggregated (e.g., concatenated, etc.) and/or otherwise utilized (e.g., leveraging voting algorithms to select from the plurality of candidates from the plurality of representations).

However, the search model 400 can be otherwise configured.

In variants, the set of molecule generation models 500 functions to generate candidate molecules based on a molecule and/or molecular fragment (e.g., moiety, chiral center(s), backbone, molecular subset, etc.) of interest. In some variants, the molecule generation model(s) can perform hit expansion for a molecule and/or molecular fragment of interest by generating new molecules based on the molecule and/or molecular fragment of interest. The molecule generation model(s) can receive, as input, a SMILES string, an embedding (e.g., determined from the representation model 300), and/or any other suitable data representation (e.g., of the hit molecule). The molecule generation model(s) 500 can produce, as output, a list of new molecules (e.g., SMILES strings, molecular formulas, molecular graphs, molecule names such as IUPAC preferred names, etc.).

The molecule generation model(s) can include pre-trained models, transformers, diffusion models (e.g., 2D diffusion models, 3D diffusion models, etc.), language models, cheminformatic models, free text models, neural networks (e.g., recurrent neural networks, graph neural networks, MLP, etc.), rules, heuristics, and/or any other models. In a specific example, the system can include an ensemble and/or plurality of molecule generation models. Within the ensemble of molecule generation models, the models can be the same or different. For example, each molecule generation model can have different structure and/or architectures, trained and/or tuned differently, trained and/or tuned using different data, utilize different feature spaces and/or embeddings, and/or otherwise differ. In variants, utilizing an ensemble of molecule generation models can include passing a hit molecule (e.g., SMILES string) as input to each molecule generation model of the ensemble and aggregating (e.g., concatenating, etc.) the lists of generated molecules from each molecule generation model. In some variations, the molecule generation model(s) can be directed. In an example of a directed model (such as GPT2ZINC), the directed molecule generation model can build off specific points (e.g., molecules, molecular fragments, hits, etc.) from a seed compound (e.g., to make specified changes based on target properties). In another variation, the molecule generation model(s) can act as a pharmacophore-based generator where one or more input compounds (e.g., hits) can be used to generate a pharmacophore and subsequently generate new compounds based on that pharmacophore.

The molecule generation model(s) can be trained and/or tuned based on experimental binding measurements, computationally predicted binding affinities, molecular property data, structural or conformational data, known ligand-RNA interaction data (e.g., binding assays, binding affinities, etc.), literature-derived annotations, high-throughput screening results, expert-curated datasets, simulated docking scores, and/or any other data suitable for supervising or refining the similarity predictions. For example, training the machine learning model can include using data labelled based on functional information, molecular information, structural information, and/or any other information. In these variants, the machine learning model can be trained to generate molecules based on function, chemistry, structure, and/or any other properties.

However, the set of molecule generation models 500 can be otherwise configured.

In variants, the system can optionally include a set of property prediction models 600, which functions to predict properties of the candidate molecules, generated molecules, and/or known molecules. The predicted properties can include functional properties, structural properties, chemical properties, and/or any other suitable properties. Functional properties can include probability of binding to a specific RNA motif, binding affinity to RNA sequences or structures, RNA selectivity, inhibition or activation of RNA function, modulation of RNA-protein interactions, selectivity, potency, off-target effects, and/or other properties. Structural properties can include three-dimensional molecular conformation, torsion angles, bond lengths, bond angles, ring strain, molecular flexibility, secondary structure propensity, and/or other geometric or topological features. Chemical properties can include molecular weight, logP, polar surface area, hydrogen bond donor/acceptor count, rotatable bonds, aromaticity, formal charge, electronegativity, LogD, solubility, permeability, stability, plasma protein binding, volume of distribution, half-life, oral bioavailability, metabolites, hERG Binding, Ames mutagenicity, cytotoxicity, CYP inhibition/induction, kinome panel interactions, docking predictors, and/or any other physicochemical properties (e.g., Absorption, Distribution, Metabolism, Excretion, and Toxicity (ADMET) properties). However, the set of property prediction models can determine any suitable properties. The property prediction model can include a machine learning model (e.g., diffusion model, transformer, neural network, etc.), a statistical model, a probabilistic model, a physics-based model, a simulator, a hybrid model, and/or any other suitable model.

In a first variant, a property prediction model can include a probability of binding model that predicts the probability a molecule and/or molecular fragment will bind to a specific RNA motif. In a second variant, a property prediction model can include a diffusion model that predicts a 3D structure of a molecule and/or molecular fragment. In a third variant, a property prediction model can include a model that predicts how accessible and/or synthesizable a molecule and/or molecular fragment is. However, a property prediction model can include any suitable models.

The property prediction model can receive a molecule representation (e.g., SMILES string, embedding, feature vector, etc.), target RNA motif, and/or any other data as input. In variants in which the property prediction model predicts a behavior and/or relationship between a molecule and an RNA motif, the property prediction model can receive both a molecule representation and an RNA representation. For example, a prediction model can use both molecule embeddings and/or RNA embeddings to predict a binding probability. In variants, predicting a probability of binding can be performed according to the method and system disclosed in U.S. application Ser. No. 17/671,391, filed 14 Feb. 2022, titled “MACHINE LEARNING PIPELINE FOR PREDICTION OF SMALL MOLECULE BINDING TO COMPLEX MOLECULES”, which is incorporated in its entirety by this reference. In another variant, predicting a probability of binding can be performed in a manner as described in Appendix A.

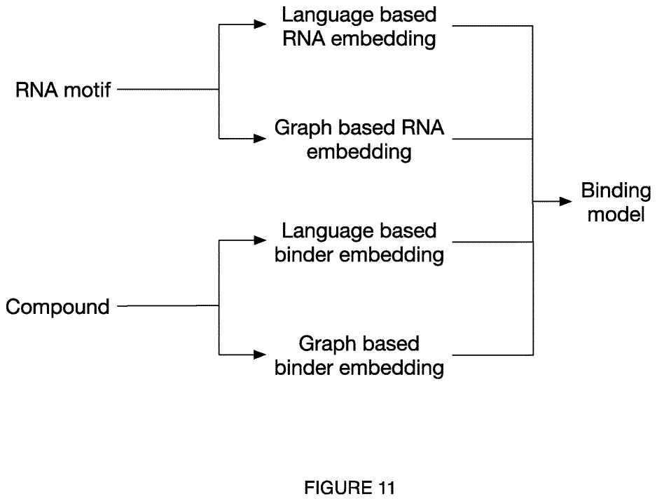

In a specific example, a property prediction model (e.g., binding model) can determine and/or receive sequence and/or structure embeddings for an RNA motif (but preferably without requiring a 3D structure for the RNA motif) and embeddings for a molecule SMILES string. In some variants (as shown for example in FIG. 11), embeddings can include both language-based (e.g., SMILES, SMARTS, mol2vec, fasttext, BERT, etc.) and graph-based (e.g., GIN_cp, GIN_im, etc.) embeddings. For example, the model can receive a language-based embedding for the sequence of an RNA motif, a graph-based embedding for the sequence of an RNA motif, a language-based embedding for the structure of an RNA motif, a graph-based embedding for the structure of an RNA motif, a language-based embedding of a SMILES string, and a graph-based embedding of a SMILES string. However, a prediction model can receive any suitable input (e.g., alternative embeddings such as cheminformatics fingerprint like ECFP6, MACCS, WHALES, Morgan fingerprints, etc.; structural fingerprint such as CCS, custom fingerprints, etc. ; chemical and/or topological properties such as Physchem calculated properties, Mordred, GeminiMol, UniMol, ECFPs, etc.; etc. where the alternative embedding can be used in addition to the language-based and graph-based embedding or can be used in place of either or both of the language-based and graph-based embedding), inclusive of a single input embedding (e.g., only a language-based embedding, only a graph-based embedding, only an alternative embedding).

However, the set of property prediction models 600 can be otherwise configured.

In variants, the embedding layers of a trained search model 400, molecule generation model 500, or property prediction model 600 can be extracted and utilized as a representation model 300. In these variants, a representation model and a prediction model can be trained end-to-end, such that the representation model is an encoder and the prediction model is a decoder. In other variants, the prediction models can utilize the embedding layers of a representation model 300 as initial input processing layers. In these variants, the representation model 300 can be trained separately from the molecule generation model, search model, or property prediction model.

In variants, the evaluation module 700 functions to evaluate (e.g., rank, etc.) a set of candidate molecules based on an optimization object. For example, the evaluation module may evaluate and/or rank a list of candidate molecules based on functional properties, chemical properties, structural properties, synthesizability, accessibility, cost, safety, stability, and/or any other suitable properties. Specific examples of properties that can be used for evaluation can include score for a predicted probability of the candidate molecule binding to an RNA target (e.g., any RNA, a target RNA structure class, a specific RNA sequence, etc.), a score for a predicted probability of the candidate molecule having a target functional impact (e.g., splicing modulation, translation modulation, etc.), docking scores (e.g., determined using a computationally generated 3d crystal structure), alerts (e.g., Brenk alerts, Derek alerts, alerts determined any other trained model, etc.), molecular weight, heavy atom count, molar refractivity, refractive index, molar volume, Log P, LogD, XLogP, WlogP, MLogP, pKa, number of formal charges (pH 7), Log S, hydrogen bond acceptor count, hydrogen bond donors count, heteroatom count, fraction sp3 carbons, aromatic ring count, property forecast index, hydrogen bond acceptor strength, hydrogen bond donor strength, polar surface area, topological polar surface area, percent polar surface area, total surface area, CNS MPO, predicted permeability, BBB penetration, sites of metabolism, passive absorption, BCS class, rotatable bonds, molecular shape index, molecular flexibility, molecular complexity, molecular volume, number of stereocenters, heteroaromatic ring count, saturated ring count, alkylamine count, arylamine count, aromatic nitrogen count, basic nitrogen count, acidic Oxygen count, drug likeness (e.g., Lipinski Rof5, Ghose, Veber, Egan, Muegge, QED, etc.), bioavailability Score, PAINS, lead-likeness, synthetic accessibility, pharmacophore methods, ligand methods, ADMET properties, other cheminformatics measures, and/or any other properties. The evaluation can be performed based on a single property, a plurality of properties, and/or any suitable number of properties. In variants, the ranked list can be used to prioritize candidate molecules for further testing. However, the evaluation module can otherwise function.

The evaluation module 700 can receive, as input, a hit expansion list (e.g., an aggregated list, a concatenated list, complete list, etc.) of candidate molecules (e.g., determined based on the hit using a search and/or generation molecule). The evaluation module can produce as output an evaluation of the list. The evaluation can include a ranking and/or ranked list, a scoring, a condensed list, a recommendation, determining and/or alerting users of less attractive molecules (e.g., based on risks, accessibility, etc.), and/or any other suitable evaluation. In variants, a ranked list and/or condensed list size can be user-defined, pre-determined, and/or otherwise defined. For example, a user can request the evaluation module to produce a ranked list of 20 molecules, 50 molecules, 100 molecules, 200 molecules, and/or any suitable number of molecules.

In variants, the evaluation can be determined based on at least one objective. The objectives can be user defined, pre-determined, determined based on a set of rules, and/or otherwise determined. In an example, objectives can include maximizing a given property, minimizing a given property, a target value for a given property, a threshold property value (e.g., below the threshold value or above the threshold value), absence of an alert, a combination thereof, and/or any other objective. In an example, evaluating the candidate molecules can include ranking the candidate molecules based on multiple objectives (e.g., using multi-parametric ranking). In a specific example, the multi-parametric ranking method can be or include weighted ranking methods, multi-objective optimization, Pareto ranking, analytic hierarchy process, outranking methods, utility theory-based methods, fuzzy ranking methods, and/or multi-parametric ranking methods.

In variants, the evaluation module can perform a plurality of separate evaluations. For example, a list of candidate molecules can be evaluated based on a first set of properties and second set of properties. The evaluations can then be aggregated, averaged, combined, weighted, cross-referenced, and/or otherwise utilized. In these variants, the evaluations can be performed in sequence, in parallel, and/or otherwise performed.

However, the evaluation module 700 can be otherwise configured.

However, the system can be otherwise configured.

### 5. Method

As shown in FIG. 1, the method can include: optionally determining RNA binding data S100; identifying a molecule and/or fragment of interest S200; and determining candidate molecules based on the molecule and/or fragment of interest S300. The method can optionally include evaluating the candidate molecules S400 and/or any other suitable steps.

All or portions of the method can be performed for each of a set of molecules of interest (e.g., query molecules, seed molecules, etc.), for each of a set of molecules in a database, and/or otherwise performed. All or portions of the method can be performed in real time (e.g., responsive to a request), iteratively, concurrently, asynchronously, periodically, and/or at any other suitable time. All or portions of the method can be performed automatically, manually, semi-automatically, and/or otherwise performed.

### 5.1. Determining RNA Binding Data S100

Determining RNA binding data S100 functions to collect RNA binding data for a set of small molecules. S100 can be performed continuously, in-response to a request, periodically, intermittently, or at any suitable frequency. For example, RNA binding data can be continuously collected to create a larger database of RNA binding data. In variants, RNA binding data can be collected over a course of days, weeks, months, years, and/or any other suitable duration, to determine a large database of RNA binding data.

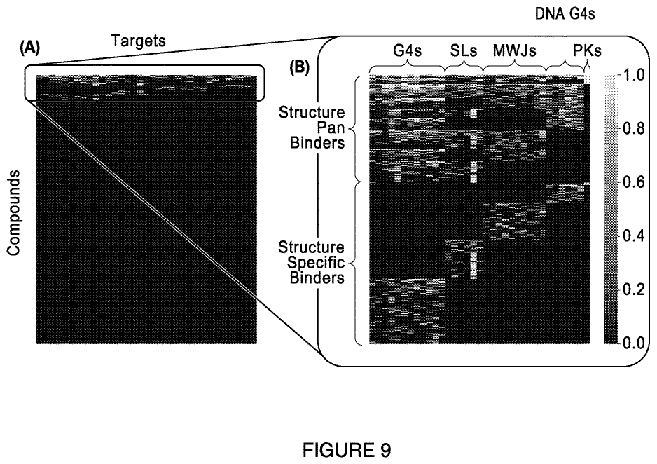

The RNA binding data can include data from one or more assays evaluating interactions between molecules and RNA. An example is shown in FIG. 9. The molecules are preferably small molecules and/or fragments of molecules, but can alternatively be any other suitable molecules. In an example, the RNA binding data can include data evaluating whether binding occurs, the functional outcome of the binding (e.g., modulating splicing, modulating translation, etc.), any other functional data (e.g., an increase and/or decrease in protein expression associated with RNA translation), and/or any other interaction properties. The RNA binding data can optionally include (e.g., as labels for assay results): RNA structure information (e.g., 2D structure, 3D structure, structure class, etc.), function information (e.g., splicing modulation information, translation modulation information, etc.), assay information (e.g., the type of assay used to collect the data), screening platform (e.g., ALIS, ASMS, SPR, etc.), context, and/or other supplemental information. In specific examples, splicing information can include the effect of a modulator (e.g., an RNA binder) on: inclusion of desired exon to enhance protein expression, inclusion of a poison exon to inhibit protein expression, and/or skipping of an exon to alter the nature of the protein expressed. In specific examples, translation information can include the effect of a modulator (e.g., an RNA binder) on: reduction and/or prevention of the binding of a protein to the targeted RNA region (e.g., RNA motif), leading to inhibition of translation; a change in binding strength of a protein to the targeted RNA region, leading to enhancement of translation (and/or vice versa for both); reduction and/or prevention of association of an miRNA with the target RNA region leading to enhancement of translation and increased expression of the protein of interest.

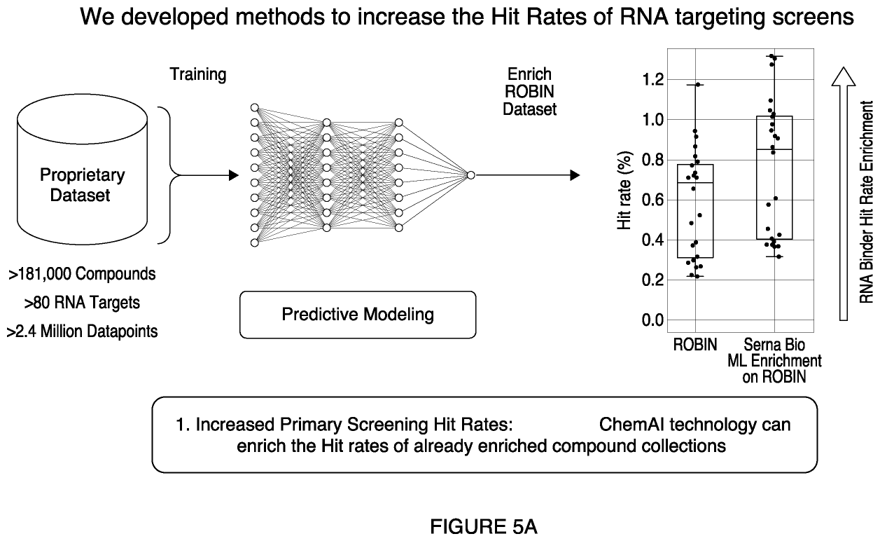

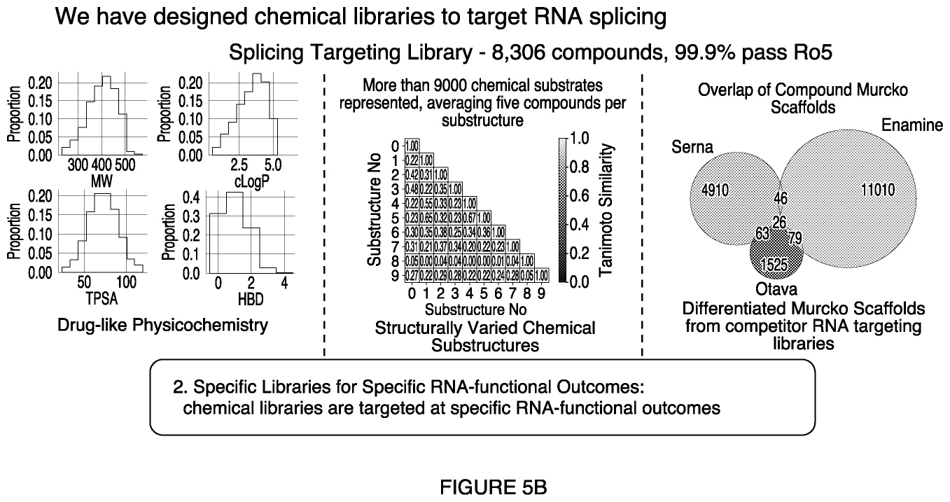

The RNA binding data can optionally include dataset(s) tailored for structure (e.g., for a specific RNA structure class), function, and/or any other supplemental information. In a first example, the RNA binding data can include data for interactions between the set of molecules and a specific structure target (e.g., multiway junction, stem loop, G-quadruplex, hairpin loops, external loops, internal loops, multihelix, junction loops, bulge loops, multiple branch loops, pseudoknots, etc.). In specific examples, the RNA binding data can include a multiway junction targeting library, a stem loop targeting library, a G-quadruplex targeting library, a hairpin loop targeting library, an external loop targeting library, an internal loop targeting library, a multihelix targeting library, a junction loop targeting library, a bulge loop targeting library, a multiple branch loop targeting library, a pseudoknot targeting library, and/or any other structure targeting library. In a second example, the RNA binding data (e.g., where the binding of the molecule affects the RNA function) can include data for molecule-RNA interactions with respect to a specific function target (e.g., translation, splicing, etc.). In specific examples, the RNA binding data can include a translation targeting library, a splicing targeting library and/or any other function targeting library. An example is shown in FIG. 5B.

However, RNA binding data may be otherwise determined.

### 5.2. Identifying a Molecule and/or Fragment of Interest S200

Identifying a molecule and/or fragment of interest S200 functions to find a hit corresponding to a given target. The molecule and/or fragment of interest (e.g., the hit, the binder, etc.) can be a small molecule and/or fragment of a molecule that binds to an RNA target (e.g., a specific RNA sequence of interest, an RNA structure class of interest, etc.), results in target functional outcome (e.g., splicing modulation, translation modulation, etc.), and/or satisfies any other RNA binding target. Note, that a fragment of a molecule can refer to specific function groups, portions of a backbone of a molecule, noncontiguous functional groups (e.g., two or more functional groups without specifying how the functional groups are connected but optionally specifying a distance or orientation between the functional groups), and/or other suitable molecule fragments. The hit can optionally be a query molecule and/or fragment used to search through known compounds for potential hits (e.g., as described in S300) and/or a seed molecule and/or fragment used to generate new compounds that are potential hits (e.g., as described in S300).

In a first variant, identifying a molecule and/or fragment of interest can include identifying an experimentally determined hit (e.g., determined using RNA binding data collected in S100). For example, the molecule and/or fragment of interest can be identified via a primary screen.

In a second variant, identifying a molecule and/or fragment of interest can include computationally predicting a hit. For example, the molecule and/or fragment of interest can be determined using a biophysics simulator, a 3D structure predictor, property prediction model, a binding predictor, a physics-based model, a machine learning model, and/or any other system. For example, the molecule and/or fragment of interest can be predicted using a model trained on the RNA binding data. In an example, the molecule and/or fragment of interest can be identified based on a representation (e.g., embedding) of the RNA target (e.g., of the RNA sequence, of the RNA structure, etc.), a representation (e.g., embedding) of the molecule and/or fragment of interest (e.g., SMILES string), and/or any other information.

In a specific example of the variant, a molecule and/or fragment of interest can be determined using a machine learning binding predictor model. A ML binding predictor model can predict a probability of binding based on a set of embeddings associated with the molecule and/or fragment (e.g., language-based embeddings, graph-based embeddings, etc.) and a set of embeddings associated with the RNA target (e.g., language-based embeddings, graph-based embeddings, sequence embeddings, structure embeddings, etc.).

However, the molecule and/or fragment of interest may be otherwise determined.

### 5.3. Determining Candidate Molecules Based on the Molecule and/or Fragment of Interest S300

Determining candidate molecules based on the molecule and/or fragment of interest S300 functions to find other molecules that are potential hits. For example, S300 can function to perform a hit expansion using the molecule and/or fragment of interest as the reference hit.

In a first variant, the candidate molecules can include known molecules, determined based on the chemical structure of the molecule and/or fragment of interest (e.g., a query molecule). In an example, determining the candidate molecules can include using a search model that outputs the candidate molecules (e.g., molecules identified as similar to the molecule and/or fragment of interest) based on the molecule and/or fragment of interest and/or associated information (e.g., molecule identifier, SMILES string, chemical structure, scaffold, etc.).

In a first embodiment of the first variant, determining candidate molecules can include identifying molecules based on a scaffold of the molecule and/or fragment of interest (e.g., using a scaffold-based search). The scaffold of the molecule and/or fragment of interest can be predetermined (e.g., Murcko scaffold), automatically determined (e.g., using a model), user-defined, and/or otherwise determined. In a specific example, the search model performs a search of a molecule library using the scaffold, wherein inputs to the model can be the molecule and/or fragment of interest and/or the scaffold. The search can optionally use a tree-based similarity search.

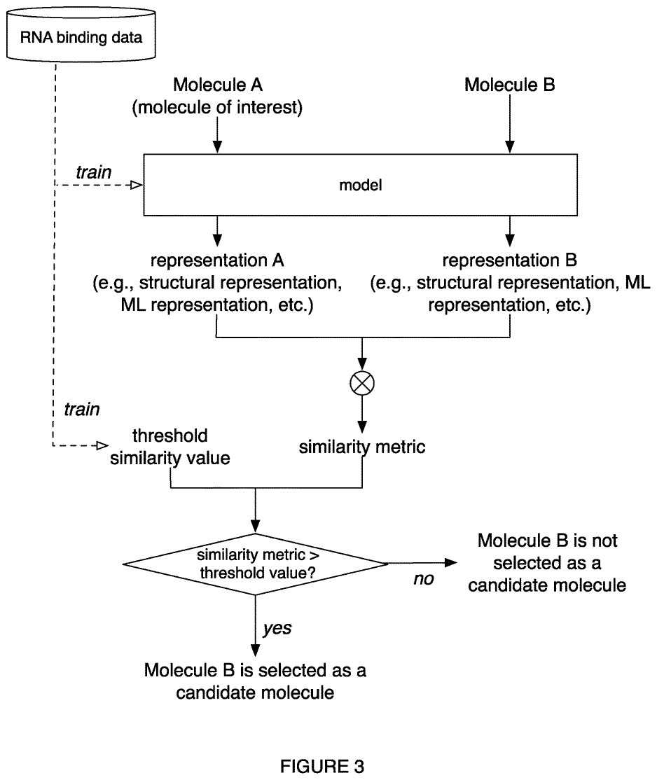

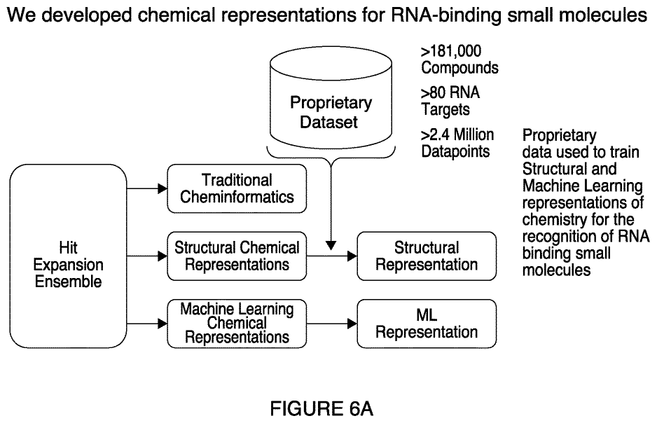

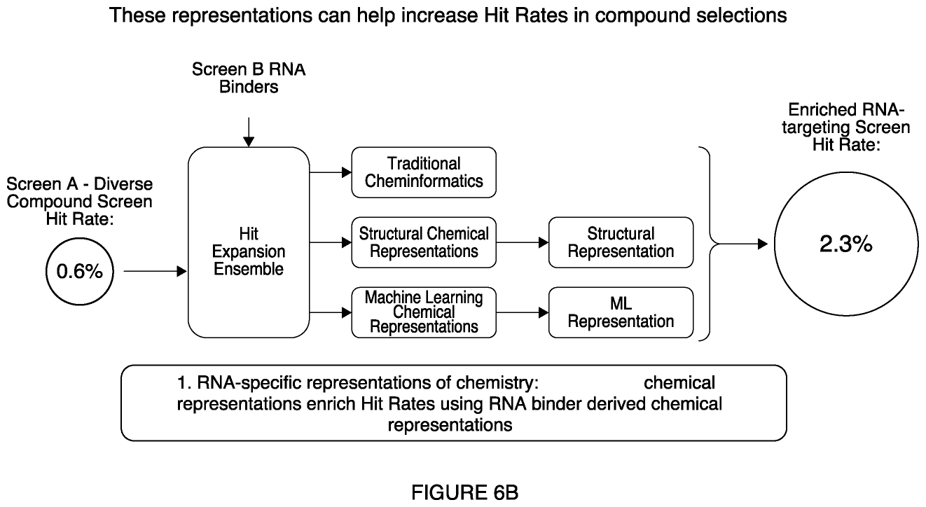

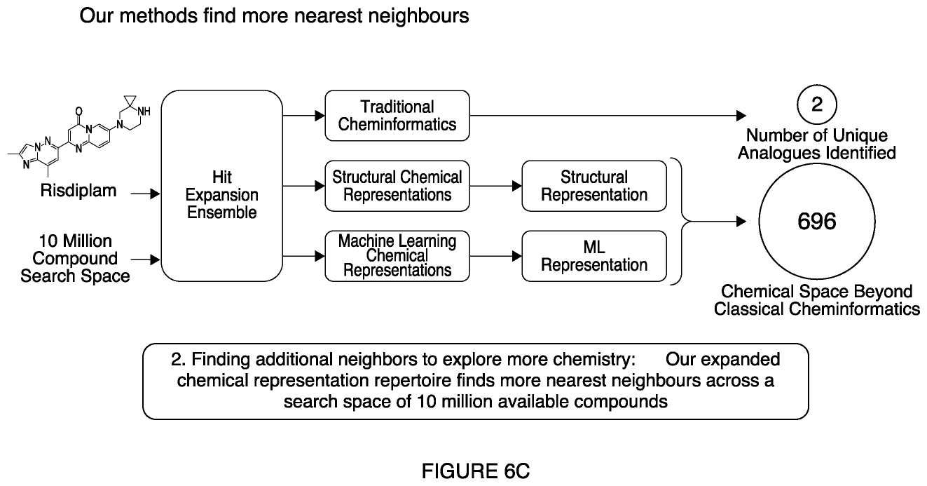

In a second embodiment of the first variant, determining candidate molecules can include determining a representation (e.g., fingerprint) based on the molecule and/or fragment of interest, wherein the candidate molecules are determined based on the representation. Examples are shown in FIG. 3 and FIGS. 6A-6C. In an example, the search model can include a representation model that outputs the representation of the molecule and/or fragment of interest within a latent space, based on the molecule and/or fragment of interest (e.g., based on a SMILES string for the molecule and/or fragment of interest). The representation of the molecule and/or fragment of interest can be a structural representation, a machine learning representation (e.g., an embedding), a physiochemical representation, and/or any other representation.

In a first example, the representation model can include a machine learning encoder that outputs an embedding within a latent space. In a second example, the representation model can include a structure-based model that determines a structural representation (e.g., a binary fingerprint) using a set of predetermined rules and/or heuristics. Specific examples of representations can include physiochemical descriptors (e.g., Mordred), machine learning representations of chemistry (e.g., ChemBERTa, etc.), binary fingerprints (e.g., such as MACCS keys), and/or any other chemistry-based representation.

The search model can perform a search within the latent space to identify candidate molecules that are similar to the molecule and/or fragment of interest. For example, the search model can: determine a similarity metric based on representations for the molecule and/or fragment of interest and a given molecule, and compare the similarity metric to a threshold similarity value to determine whether the given molecule is selected as a candidate molecule. The similarity metric can include: a distance within the latent space, a dot product, a ratio, Tanimoto coefficient (also called the Jaccard index and the Tanimoto index), Dice index (also called Dice-Sørensen coefficient), Cosine coefficient (also referred to a cosine similarity), Soergel distance, Euclidean distance, Manhattan distance, Minkowski distance, Chebyshev distance, Edit distance, Levenshtein distance, Lee distance, Hamming distance, Jaro distance, dynamic time warping, Bhattacharyya distance, Hellinger distance, and/or any other comparison between representations.

In a first example, the candidate molecules can be the molecules within a threshold distance of the molecule and/or fragment of interest in the latent space (e.g., within a threshold radius around the molecule and/or fragment of interest). In variants, determining a distance in the latent space can include computing cosine similarity, Euclidean distance, Manhattan distance, Mahalanobis distance, Chebyshev distance, Minkowski distance, Hamming distance (e.g., for binary fingerprints), Jaccard distance, Tanimoto distance, kernel-based similarity measures, learned similarity metrics (e.g., metric learning models), graph edit distance, embedding-space dot product similarity, and/or any other similarity or distance metric.

In a second example, the candidate molecules can be the nearest neighbors (e.g., top 100 closest molecules) to the molecule and/or fragment of interest in the latent space. In a specific example, the model can use a k-nearest neighbours approach to identify the k nearest neighbors to the molecule and/or fragment of interest within a given latent space (e.g., where k is determined by the user). In other examples, the model can use hierarchical clustering (e.g., agglomerative clustering, divisive clustering), density-based clustering (e.g., DBSCAN, HDBSCAN), centroid-based clustering (e.g., k-means, k-medoids), spectral clustering, Gaussian mixture models, affinity propagation, mean-shift clustering, community detection methods, approximate nearest neighbor algorithms (e.g., locality-sensitive hashing, hierarchical navigable small world graphs), and/or any other clustering or neighborhood discovery method.

The threshold similarity value can be: predetermined (e.g., a Tanimoto coefficient greater than 0.7), learned, user defined, experimentally determined (e.g., using data collected via S100), and/or otherwise determined. In an illustrative example, the threshold similarity value can be determined by: performing a screening assay on set of molecules; iteratively, for each of a set of threshold similarity values, selecting a subset of molecules in the set of molecules using the respective threshold similarity value; and evaluating the change in the hit rate (e.g., the change in the percent of molecules in the subset of molecules that pass the screening assay) as the threshold similarity value increases. In a specific variant, the threshold similarity value can be selected to minimize false positives, maximize accuracy, and/or achieve any other suitable objective. In variants, the threshold similarity value can be dynamically adjusted based on characteristics of the latent space, the density of molecules within a region of the latent space, the distribution of similarity scores across a reference dataset, and/or a target number of candidate molecules to retrieve. In other variants, the threshold similarity value can be optimized using cross-validation, Bayesian optimization, grid search, reinforcement learning, and/or any other parameter optimization technique.

The search model and/or component thereof (e.g., the representation model, the latent space, the similarity threshold value, etc.) can be trained (e.g., tuned) using RNA binding data (e.g., collected via S100). In a specific example, the search model and/or components thereof can be tuned via one or more epochs using the RNA binding data (e.g., from S100) as training data. In specific examples, the number of epochs used can be: at least 1, at least 2, at least 3, at least 4, at least 5, and/or any other suitable number of epochs. In an example, the tuned latent space for the generation model can be used as the latent space for the search model (e.g., as described in the second variant, below).

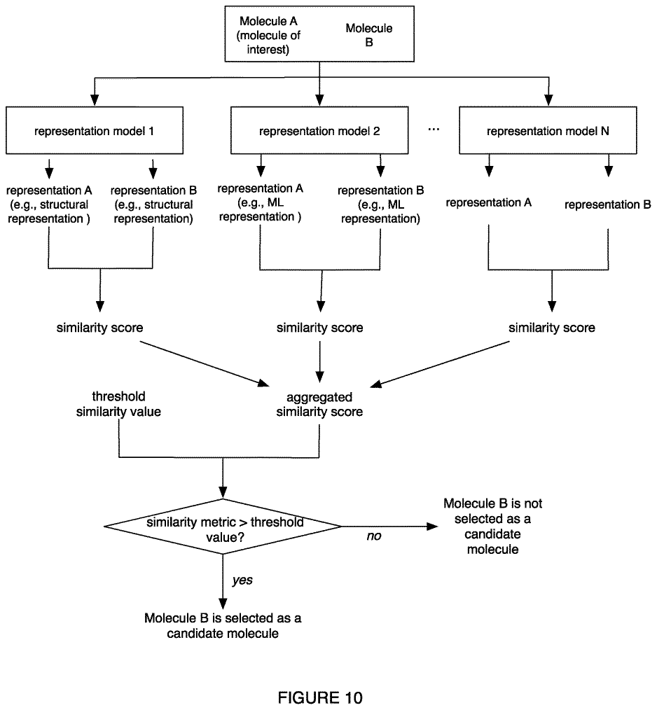

In variants, the search model can include a set and/or ensemble of representation models. In these variants, a similarity score between each candidate molecule and/or molecular fragment and the hit can be determined based on each representation of a set of representation models, as shown for example in FIG. 10. For example, a first representation model can determine a fingerprint-based representation (e.g., a circular fingerprint), a second representation model can determine a graph-based embedding, and a third representation model can determine a language-model embedding derived from a SMILES string. Similarity scores derived from each representation can capture different aspects of molecular similarity (e.g., structural similarity, functional similarity, or contextual similarity). The similarity score can be averaged, summed, weighted averaged, and/or otherwise aggregated. The aggregated similarity score can be compared to a threshold similarity score.

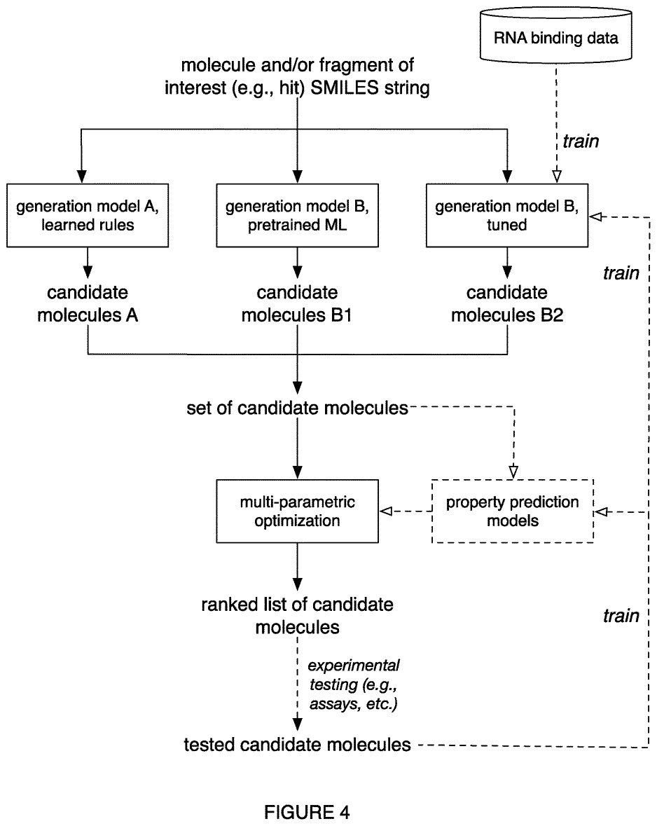

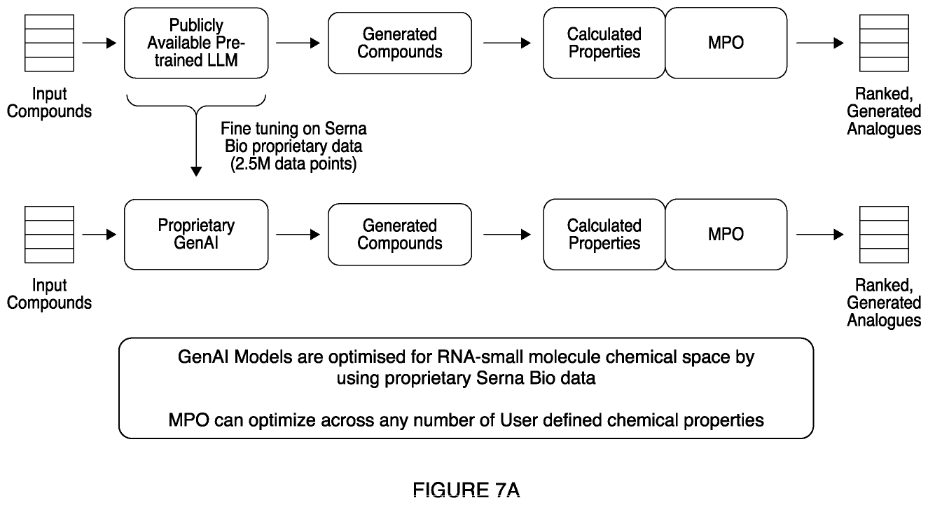

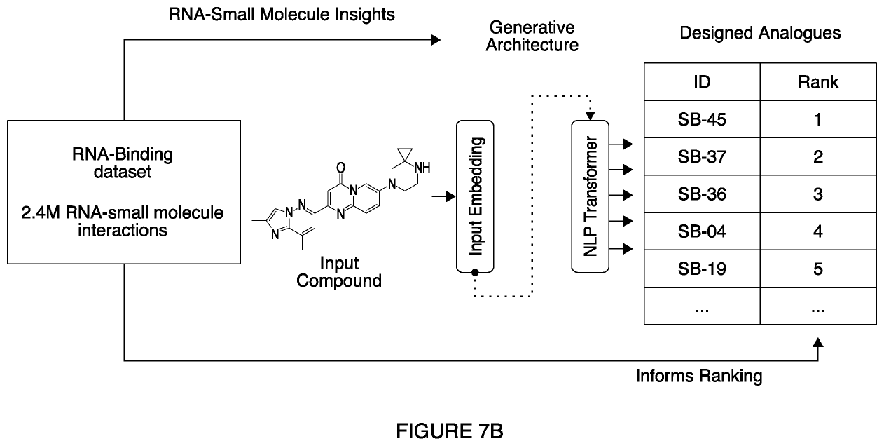

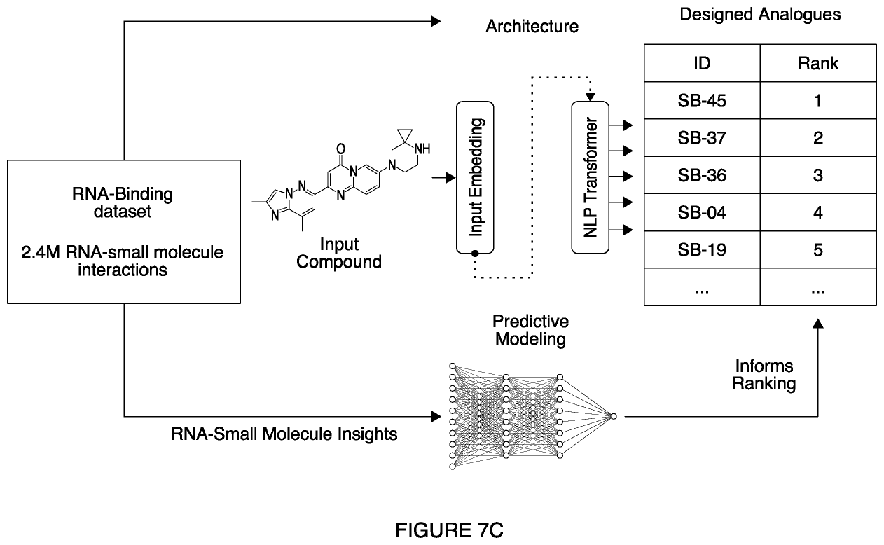

In a second variant, the candidate molecules can include newly generated chemical structures, determined based on the chemical structure of the molecule and/or fragment of interest (e.g., a seed molecule). Examples are shown in FIG. 4, FIGS. 7A-7C, and FIGS. 8A-8C.

In an example, the candidate molecules can be generated using one or more generation models. In variants, the system can include a set and/or ensemble of generation models, where each generation model generates a distinct set of candidate molecules. However, in other variants, the system can include a single generation model.

The input to a generation model can include a Simplified Molecular Input Line Entry System (SMILES) string for the molecule and/or fragment of interest and/or any other representation of the chemical structure of the molecule and/or fragment of interest. The output from a generation model can include generated SMILES strings for one or more candidate molecules and/or any other representation of the chemical structure of the one or more candidate molecules. In a specific example, multiple generation models can be ensembled in parallel, wherein the output candidate molecules of each generation model are aggregated to form a superset of the generated candidate molecules. In another specific example, the output(s) of one generation model in an ensemble of generation models can be used as the starting point (e.g., input) for other generation models in the ensemble (e.g., in a feedback loop).

In a first example, the generation model can include a rule-based model performing chemically valid transformations. In a specific example, the generation model can be a matched molecular pairs (MMP) model.

In a second example, the generation model can include a large language model. In a specific example, the generation model can include a masked language model (e.g., a BERT model trained on SMILES strings). In a specific example, the generation model can mask part(s) of the input SMILES string for the molecule and/or fragment of interest and predict a valid replacement for the masked section(s).

In a third example, the generation model can include a graph-based model. In variants, the graph-based model can operate directly on molecular graphs, wherein atoms correspond to nodes and bonds correspond to edges. The model can iteratively construct candidate molecules by adding nodes, edges, and/or substructures while enforcing chemical validity constraints (e.g., valence constraints). Examples of graph-based models can include graph neural networks, graph variational autoencoders, graph generative adversarial networks, and graph reinforcement learning models configured to generate chemically valid molecular structures.

In a fourth example, the generation model can include a transformer model. In variants, the transformer model can be trained to generate molecular representations (e.g., SMILES strings, SELFIES strings, or other sequence-based representations) using attention mechanisms. The transformer model can condition generation on the molecule and/or fragment of interest, on a target RNA structure class, on a target functional outcome, and/or on other contextual inputs. In specific examples, the transformer model can generate candidate molecules via autoregressive sequence generation, conditional generation, or masked token prediction.

In a fifth example, the generation model can include a free text model. In variants, the free text model can generate candidate molecules based on natural language prompts describing desired molecular characteristics, RNA targets, structural motifs, functional outcomes, and/or other constraints. The free text model can translate the textual description into one or more molecular representations (e.g., SMILES strings) corresponding to candidate molecules predicted to satisfy the described properties.

In a sixth example, the generation model can include a cheminformatic model. In variants, the cheminformatic model can generate candidate molecules using rule-based transformations, fragment recombination, scaffold hopping methods, combinatorial chemistry enumeration, reaction-based enumeration, retrosynthesis-based generation, and/or library enumeration techniques.

In a seventh example, the generation model can include a diffusion model (e.g., 2D diffusion model, 3D diffusion model, etc.). In variants, the diffusion model can iteratively generate candidate molecular structures by progressively denoising a random initial representation. The diffusion process can operate in two-dimensional molecular graph space, three-dimensional atomic coordinate space, and/or latent embedding space. In specific examples, the diffusion model can be conditioned on the molecule and/or fragment of interest, target RNA structural features, predicted binding interactions, and/or desired molecular properties

The generation model(s) can be pretrained and/or tuned (e.g., retrained) models. In a first specific example, the generation model can be pretrained on data not specific to RNA binding. In a second specific example, the generation model can be a pretrained model (e.g., the model as described in the first specific example that is subsequently tuned on general RNA binding data. In a third specific example, the generation model can be a pretrained model (e.g., the model as described in the first specific example) that is subsequently tuned on structure-specific RNA binding data (e.g., a stem loop targeting library, a G-quadruplex targeting library, a multi-way junction targeting library, etc.) and/or function-specific RNA binding data (e.g., a translation targeting library, a splicing targeting library, etc.). In a fourth specific example, the generation model can be a tuned model (e.g., as described in specific example 2) that is subsequently tuned on structure-specific and/or chemical series specific RNA binding data (e.g., a stem loop targeting library, a G-quadruplex targeting library, a multi-way junction targeting library, etc.) and/or functional data (e.g., a translation targeting library, a splicing targeting library, etc.).

Generation models can optionally be tuned using RNA binding data (e.g., determined via S100). In an example, generation models can be tuned via one or more epochs, using the RNA binding data as training data. In specific examples, the number of epochs used can be: at least 1, at least 2, at least 3, at least 4, at least 5, and/or any other suitable number of epochs. In a specific example, a first generation model can be a pretrained model tuned using a first number of epochs, and a second generation model can be a pretrained model tuned using a second number of epochs, wherein the second number of epochs is greater than the first number of epochs. In an example, the (tuned) latent space for a generation model can be used as the latent space for the search model (e.g., as described in the first variant).

However, the candidate molecules may be otherwise determined.

Downstream processes using the candidate molecules can include: validating the candidate molecules as confirmed hits using assays (e.g., primary screens, secondary screens, etc.), evaluating the candidate molecules as leads for drug discovery (e.g., via S400), using the candidate molecules to identify other hits (e.g., via another iteration of S300), adding the candidate molecules to a chemical library, and/or any other suitable uses. In an example, the candidate molecules can be used to build an enriched chemical library. An example is shown in FIG. 5A. In a specific example, the candidate molecules determined using one or more methods described above can be aggregated to form an enriched chemical library of molecules more likely to contain hits for a given target relative to an unenriched library of molecules. The chemical library can optionally be enriched for a given structure target (e.g., multiway junction, stem loop, G-quadruplex, hairpin loops, external loops, internal loops, multihelix, junction loops, bulge loops, multiple branch loops, pseudoknots, etc.) and/or function target (e.g., splicing, translation, etc.).

However, the candidate molecules can be otherwise used.

### 5.4. Evaluating the Candidate Molecules S400

Evaluating the candidate molecules S400 functions to identify which molecules to acquire (e.g., synthesize, purchase, etc.) for experimental testing (e.g., screening), drug development, and/or other downstream processes. S400 can be performed using the evaluation module.

The candidate molecules can be evaluated with respect to one or more parameters and one or more objectives (e.g., one objective for each parameter). The parameters preferably include more than 2 parameters (e.g., at least 2 parameters, at least 5 parameters, at least 10 parameters, etc.), but can alternatively be a single parameter. Values for the parameters (e.g., for a given candidate molecule) can be determined experimentally, measured, computationally predicted, retrieved from a database, and/or otherwise determined.

In an example, the parameters can include: a score for a predicted probability of the candidate molecule binding to an RNA target (e.g., any RNA, a target RNA structure class, a specific RNA sequence, etc.), a score for a predicted probability of the candidate molecule having a target functional impact (e.g., splicing modulation, translation modulation, etc.), docking scores (e.g., determined using a computationally generated 3d crystal structure), alerts (e.g., Brenk alerts, Derek alerts, alerts determined any other trained model, etc.), molecular weight, heavy atom count, molar refractivity, refractive index, molar volume, Log P, LogD, XLogP, WlogP, MLogP, pKa, number of formal charges (pH 7), Log S, hydrogen bond acceptor count, hydrogen bond donors count, heteroatom count, fraction sp3 carbons, aromatic ring count, property forecast index, hydrogen bond acceptor strength, hydrogen bond donor strength, polar surface area, topological polar surface area, percent polar surface area, total surface area, CNS MPO, predicted permeability, BBB penetration, sites of metabolism, passive absorption, BCS class, rotatable bonds, molecular shape index, molecular flexibility, molecular complexity, molecular volume, number of stereocenters, heteroaromatic ring count, saturated ring count, alkylamine count, arylamine count, aromatic nitrogen count, basic nitrogen count, oxygen count, drug likeness (e.g., Lipinski Rof5, Ghose, Veber, Egan, Muegge, QED, etc.), bioavailability Score, PAINS, lead-likeness, synthetic accessibility, 3D structure, other cheminformatics measures, and/or any other physiochemical properties.

In variants, the parameters and/or subsets of the parameter can be determined and/or predicted using property prediction models. For example, the probability of the candidate molecule binding to an RNA target can be determined using a binding model. In a second example, a 3D structure can be predicted using a 3D diffusion model. In variants, the 3D structure can be predicted using a machine learning structure prediction model trained on molecular conformations, a molecular mechanics force field simulation, a Monte Carlo conformational search, molecular dynamics simulations, normal mode analysis, and/or any other structure prediction method. In a third example, a synthetic accessibility score can be predicted using a machine learning model.

The objectives are preferably user defined, but can alternatively be otherwise determined. In an example, objectives can include: maximize a given parameter, minimize a given parameter, a target value for a given parameter, a threshold parameter value (e.g., below the threshold value or above the threshold value), absence of an alert, maximize a set of parameters, minimize a set of parameters, a combination thereof, and/or any other objective.

Evaluating the candidate molecules can include scoring each candidate molecule, ranking the candidate molecules, selecting a subset of the candidate molecules, a combination thereof, and/or any other suitable evaluation method. In an example, evaluating the candidate molecules can include ranking the candidate molecules using multi-parametric ranking. In a specific example, the multi-parametric ranking method can be or include: weighted ranking methods, multi-objective optimization, Pareto ranking, analytic hierarchy process, outranking methods, utility theory-based methods, fuzzy ranking methods, and/or multi-parametric ranking methods.

In a variant, ranking the candidate molecules can include performing a multi-step ranking. In these variants, the candidate molecules can be first ranked according to one set of objectives and/or parameters, and subsequently ranked by other objectives and/or parameters. This variant can result in multiple ranking of the candidate molecules based on the objectives and/or parameters. The multiple rankings can be aggregated, cross-referenced, and/or otherwise utilized to create a final ranked list of candidate molecules. However, other variants can include a single ranking. Ranking can be otherwise performed.

S400 can output a ranked list of the candidate molecules, the score for each candidate molecule, a subset of the candidate molecules (e.g., the top 100 candidate molecules). Examples are shown in FIG. 7A, FIG. 7B, and FIG. 7C. The output can optionally include a tag for the candidate molecule(s) that have an alert (e.g., Brenk alerts, Derek alerts, alerts determined by any other trained model, etc.). In an example, the alert can be determined using a model (e.g., expert system) trained using expert knowledge (e.g., determined by scientists) to label SMILES strings with chemical alerts based on their structures. In another example, the alerts can be determined using structural enrichment on bespoke chemical data. In another example, alerts can be generated based on a similarity between a candidate molecule and known compounds with alerts or undesirable chemistry (e.g., pan-RNA binding, pan-DNA binding, etc.). Variations can include a single alert type (e.g., generated by a single mechanism above) and/or a plurality of alert types (e.g., a combination of the above alert types).

In variants, evaluating the candidate molecules can include performing experimental tests. For example, the candidate molecules can be synthesized, acquired, and/or otherwise obtained and tested using one or more assays configured to evaluate interaction with the RNA target (e.g., binding assays, functional assays, structural assays, biochemical assays, cellular assays, phenotypic screen, etc.). The experimental evaluation can determine measured outcomes such as binding affinity, binding probability, functional modulation of the RNA target, structural interaction characteristics, toxicity, stability, and/or other experimentally observable properties. In variants, the experimentally determined dataset can be added to the database.

However, the candidate molecules may be otherwise evaluated.

The method can optionally include re-training and/or tuning the models (e.g., representation models, search models, molecule generation models, property prediction models, etc.). The models can be re-trained and/or tuned based on the evaluation determined in S400. In variants, after determining the candidate molecules, a subset of the candidate molecules can be determined (e.g., based on the evaluation, based on experimental tests, etc.). In variants, a subset of molecules can be identified (e.g., molecules demonstrating binding to the RNA motif, molecules exceeding a threshold activity value, molecules meeting specified functional criteria, etc.) and utilized to re-train, tune, and/or update models.

In variants, evaluation and/or experimental testing can be used to determine a subset of molecules to be used for training. In other variants, evaluation and/or experimental testing can be used to determine training labels. For example, experimental tests can determine binding affinity and/or other properties which can be used as ground-truth data and/or labels for training the models. In variants, both positive and negative experimental outcomes can be used as training data. For example, molecules determined not to bind the RNA motif, or molecules exhibiting low activity, can be used as negative training examples for retraining prediction models, similarity models, and/or representation models.

In some variants, the updated models can subsequently be used to generate or identify additional candidate molecules, thereby forming an iterative discovery process in which experimental results progressively improve the predictive accuracy and search capability of the models. Additionally or alternatively, generated compounds can be fed into chemical generators (e.g., search models, generative models, etc.) when necessary to design more candidation molecules (e.g., for multiparametric optimization). In variants, the subset of experimentally validated molecules can be used to adjust similarity metrics, update latent space representations, re-train prediction models, and/or guide subsequent candidate molecule generation.

In variants, the models can be updated using incremental training, online learning, batch retraining, fine-tuning, transfer learning, and/or any other model update technique. In some examples, newly generated experimental data can be periodically incorporated into a training dataset and used to update the models while preserving previously learned information.

### Specific Examples

A numbered list of specific examples of the technology described herein are provided below. A person of skill in the art will recognize that the scope of the technology is not limited to and/or by these specific examples.

1. A method comprising determining a target RNA motif; determining a hit of the target RNA motif; for each model from an ensemble of models: generating a set of potential hits based on the hit using the respective model; aggregating the sets of potential hits from each model of the ensemble of models to form an aggregated set of potential hits; and ranking the aggregated set of potential hits based on a molecular property of each potential hit in the aggregated set of potential hits; wherein the ensemble of models comprises at least one of a cheminformatics model, a language model, a transformer, or a diffusion model.

2. The method of Specific Example 1, further comprising performing a chemical binding assay for one or more potential hits from the aggregated set of potential hits, wherein the ensemble of models or models thereof are tuned based on the chemical binding assay.

3. The method of any of Specific Examples 1-2, wherein the molecular property comprises a 3D structure of the potential hit.

4. The method of Specific Example 3, wherein the 3D structures are predicted using a 3D diffusion model.

5. The method of any of Specific Examples 1-4, wherein each model of the ensemble of models receives a SMILES string of the hit.

6. The method of any of Specific Examples 1-5, wherein the molecular property comprises a binding probability of the potential hit to the RNA motif.

7. The method of Specific Example 6, wherein the binding probability for each potential hit in the aggregated set of potential hits is determined using a machine learning model that ingests an embedding of the target RNA motif and an embedding of the respective potential hit.

8. The method of any of Specific Examples 1-7, wherein ranking the aggregated set of potential hits further comprises ranking each potential hit of the aggregated set of potential hits based on a second molecular property, and aggregating the ranking based on the molecular property and the ranking based on the second molecular property.

9. The method of any of Specific Examples 1-8, wherein the hit of the RNA motif comprises a fragment of a molecule, wherein each potential hit of the aggregated set of potential hits is a complete molecule.

10. A method comprising: determining a target RNA motif; determining a hit of the target RNA motif; determining a hit latent embedding of the hit using an embedding model; and identifying potential hits by performing a search in a database of a plurality of potential hits, comprising for each potential hit in the database: determining a potential hit latent embedding in the database using the embedding model; determining a similarity score between the hit and the respective potential hit based on the hit latent embedding and the potential hit latent embedding; determining whether the respective potential hit is likely to bind with the target RNA motif based on the similarity score.

11. The method of Specific Example 10, further comprising: determining a second hit latent embedding of the hit using a second embedding model; for each potential hit in the database: determining a second potential hit latent embedding in the database using the second embedding model; determining a second similarity score between the hit and the respective potential hit based on the hit latent embedding and the potential hit latent embedding; determining whether the respective potential hit is likely to bind with the target RNA motif based on the similarity score and the second similarity score.

12. The method of any of Specific Examples 10-11, wherein determining the hit of the target RNA motif comprises: determining a target RNA embedding of the target RNA motif using an RNA embedding model; determining a hit embedding of the hit using a hit embedding model; predicting a binding affinity of the hit with the target RNA motif based on the target RNA embedding and the hit binding embedding using a binding model.

13. The method of Specific Example 12, wherein the target RNA embedding and the hit embedding each comprise graph-based embeddings and language-based embeddings.

14. The method of any of Specific Examples 12-13, further comprising performing an assay using a potential hit of the potential hits, wherein the binding model is updated based on the assay.

15. The method of any of Specific Examples 10-15, further comprising tuning the embedding model based on a binding success of the potential hits.

16. The method of any of Specific Examples 10-16, wherein determining the similarity score between the hit and each potential hit comprises computing a distance between the hit latent embedding and the potential hit latent embedding in an embedding space.

17. The method of Specific Example 16, wherein the hit latent embedding is determined based on a SMILES string of the hit.

18. The method of any of Specific Examples 10-17, further comprising ranking the potential hits based on a molecular property of each of the potential hits.

19. The method of Specific Example 18, wherein the molecule property comprises at least one of a synthetic accessibility score, a binding probability, or a 3D structure.

20. The method of Specific Example 19, wherein the 3D structure is predicted using a diffusion model.

21. A computer-implemented method, comprising: receiving a test ribonucleic acid (RNA) and a test small molecule (SMOL); generating an RNA embedding representing the test RNA, the RNA embedding generated in a first latent space; generating a SMOL embedding representing the test SMOL, the SMOL embedding generated in a second latent space; inputting the RNA embedding and the SMOL embedding into to a predictive binding model, wherein the predictive binding model is a machine-learned model trained with a plurality of training samples, each training sample comprising a binding result between an RNA and a SMOL; determining, by the predictive binding model, a binding prediction comprising a binary prediction of whether the test SMOL will bind to the test RNA; and providing, via the graphical user interface, a result that includes the binding prediction.

22. The computer-implemented method of Specific Example 21, wherein the binding prediction further comprises a binding probability that the test SMOL will bind to the test RNA.

23. The computer-implemented method of any of Specific Examples 21-22, wherein the first latent space includes one or more dimensions that represent one or more of the following: (1) the secondary structures formed by the folding of an RNA sequence, (2) the ensembled behavior of secondary structures present on a single RNA sequence, and (3) pseudoknots.

24. The computer-implemented method of any of Specific Examples 21-23, wherein the SMOL embedding is generated based on a molecular fingerprint of the candidate small molecule based on a SMILES and/or ECFP model.

25. The computer-implemented method of any of Specific Examples 21-24, wherein the predictive binding model is further trained by: generating additional training samples through in vitro screening of one or more small molecules; and training the predictive binding model with results from the in vitro screening of the additional training samples.

26. The computer-implemented method of any of Specific Examples 21-25, wherein the predictive binding model is a one-dimensional convolutional neural network or a logistic regression model.

27. The computer-implemented method of any of Specific Examples 21-26, wherein the predictive binding model is further trained by: receiving a binding result of a validation experiment between the test RNA and the test SMOL; comparing the binding result to the binding prediction; and refining the predictive binding model based on the comparison.

28. The computer-implemented method of any of Specific Examples 21-27, wherein the RNA sequence is received via the graphical user interface through a user upload of the RNA sequence or a user selection of a predefined RNA sequence.

29. The computer-implemented method of any of Specific Examples 21-28 further comprising: generating a context embedding representing a biological context of the test RNA and the test SMOL, the context embedding generated in a third latent space; wherein the context embedding is further input into the predictive binding model to determine the binding prediction.

30. The computer-implemented method of Specific Example 29, wherein the biological context comprises one or more of: presence of one or more additional RNAs; presence of disease-associated single nucleotide polymorphisms (SNPs); and another type of genetic information specific to an individual.

31. A non-transitory computer-readable medium storing instructions that, when executed by one or more processors, cause the one or more processors to perform operations comprising: receiving a test ribonucleic acid (RNA) and a test small molecule (SMOL); generating an RNA embedding representing the test RNA, the RNA embedding generated in a first latent space; generating a SMOL embedding representing the test SMOL, the SMOL embedding generated in a second latent space; inputting the RNA embedding and the SMOL embedding into to a predictive binding model, wherein the predictive binding model is a machine-learned model trained with a plurality of training samples, each training sample comprising a binding result between an RNA and a SMOL; determining, by the predictive binding model, a binding prediction comprising a binary prediction of whether the test SMOL will bind to the test RNA; and providing, via the graphical user interface, a result that includes the binding prediction.

32. The non-transitory computer-readable medium of Specific Example 31, wherein the binding prediction further comprises a binding probability that the test SMOL will bind to the test RNA.

33. The non-transitory computer-readable medium of any of Specific Examples 31-32, wherein the first latent space includes one or more dimensions that represent one or more of the following: (1) the secondary structures formed by the folding of an RNA sequence, (2) the ensembled behavior of secondary structures present on a single RNA sequence, and (3) pseudoknots.

34. The non-transitory computer-readable medium of any of Specific Examples 31-33, wherein the SMOL embedding is generated based on a molecular fingerprint of the candidate small molecule based on one or both of a SMILES model and an ECFP model.

35. The non-transitory computer-readable medium of any of Specific Examples 31-34, wherein the predictive binding model is further trained by: generating additional training samples through in vitro screening of one or more small molecules; and training the predictive binding model with results from the in vitro screening of the additional training samples.

36. The non-transitory computer-readable medium of any of Specific Examples 31-35, wherein the predictive binding model is a one-dimensional convolutional neural network or a logistic regression model.

37. The non-transitory computer-readable medium of any of Specific Examples 31-36, wherein the predictive binding model is further trained by: receiving a binding result of a validation experiment between the test RNA and the test SMOL; comparing the binding result to the binding prediction; and refining the predictive binding model based on the comparison.

38. The non-transitory computer-readable medium of any of Specific Examples 31-37, wherein the RNA sequence is received via the graphical user interface through a user upload of the RNA sequence or a user selection of a predefined RNA sequence.

39. The non-transitory computer-readable medium of any of Specific Examples 31-38, the operations further comprising: generating a context embedding representing a biological context of the test RNA and the test SMOL, the context embedding generated in a third latent space; wherein the context embedding is further input into the predictive binding model to determine the binding prediction.

40. The non-transitory computer-readable medium of Specific Example 39, wherein the biological context comprises one or more of: presence of one or more additional RNAs; presence of disease-associated single nucleotide polymorphisms (SNPs); and another type of genetic information specific to an individual.

41. A method comprising leveraging the computer-implemented processes of any of Specific Examples 21-30 or the non-transitory computer-readable medium of any of Specific Examples 31-40 for the generation, selection, characterization, tuning, refinement, and/or other processes (e.g., using as the models, using to train the models, using to evaluate the outputs of the models, using to select the models, etc.) of any of Specific Examples 1-20.

As used herein, “substantially” or other words of approximation (e.g., “about,” “approximately,” etc.) can be within a predetermined error threshold or tolerance of a metric, component, or other reference (e.g., within +/−0.001%, +/−0.01%, +/−0.1%, +/−1%, +/−2%, +/−5%, +/−10%, +/−15%, +/−20%, +/−30%, any range or value therein, of a reference).

Alternative embodiments implement the above methods and/or processing modules in non-transitory computer-readable media, storing computer-readable instructions that, when executed by a processing system, cause the processing system to perform the method(s) discussed herein. The instructions can be executed by computer-executable components integrated with the computer-readable medium and/or processing system. The computer-readable medium may include any suitable computer readable media such as RAMs, ROMs, flash memory, EEPROMs, optical devices (CD or DVD), hard drives, floppy drives, non-transitory computer readable media, or any suitable device. The computer-executable component can include a computing system and/or processing system (e.g., including one or more collocated or distributed, remote or local processors) connected to the non-transitory computer-readable medium, such as CPUs, GPUs, TPUS, microprocessors, or ASICs, but the instructions can alternatively or additionally be executed by any suitable dedicated hardware device.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

We claim:

1. A method comprising determining a target RNA motif;
determining a hit of the target RNA motif;
for each model from an ensemble of models:
generating a set of potential hits based on the hit using the respective model;
aggregating the sets of potential hits from each model of the ensemble of models to form an aggregated set of potential hits; and
ranking the aggregated set of potential hits based on a molecular property of each potential hit in the aggregated set of potential hits;
wherein the ensemble of models comprises at least one of a cheminformatics model, a language model, a transformer, or a diffusion model.

2. The method of claim 1, further comprising performing a chemical binding assay for one or more potential hits from the aggregated set of potential hits, wherein the ensemble of models or models thereof are tuned based on the chemical binding assay.

3. The method of claim 1, wherein the molecular property comprises a 3D structure of the potential hit.

4. The method of claim 3, wherein the 3D structures are predicted using a 3D diffusion model.

5. The method of claim 1, wherein each model of the ensemble of models receives a SMILES string of the hit.

6. The method of claim 1, wherein the molecular property comprises a binding probability of the potential hit to the RNA motif.

7. The method of claim 6, wherein the binding probability for each potential hit in the aggregated set of potential hits is determined using a machine learning model that ingests an embedding of the target RNA motif and an embedding of the respective potential hit.

8. The method of claim 1, wherein ranking the aggregated set of potential hits further comprises ranking each potential hit of the aggregated set of potential hits based on a second molecular property, and aggregating the ranking based on the molecular property and the ranking based on the second molecular property.

9. The method of claim 1, wherein the hit of the RNA motif comprises a fragment of a molecule, wherein each potential hit of the aggregated set of potential hits is a complete molecule.

10. A method comprising:
determining a target RNA motif;
determining a hit of the target RNA motif;
determining a hit latent embedding of the hit using an embedding model; and
identifying potential hits by performing a search in a database of a plurality of potential hits, comprising for each potential hit in the database:
determining a potential hit latent embedding in the database using the embedding model;
determining a similarity score between the hit and the respective potential hit based on the hit latent embedding and the potential hit latent embedding;
determining whether the respective potential hit is likely to bind with the target RNA motif based on the similarity score.

11. The method of claim 10, further comprising:
determining a second hit latent embedding of the hit using a second embedding model;
for each potential hit in the database:
determining a second potential hit latent embedding in the database using the second embedding model;
determining a second similarity score between the hit and the respective potential hit based on the hit latent embedding and the potential hit latent embedding;
determining whether the respective potential hit is likely to bind with the target RNA motif based on the similarity score and the second similarity score.

12. The method of claim 10, wherein determining the hit of the target RNA motif comprises:
determining a target RNA embedding of the target RNA motif using an RNA embedding model;
determining a hit embedding of the hit using a hit embedding model;
predicting a binding affinity of the hit with the target RNA motif based on the target RNA embedding and the hit binding embedding using a binding model.

13. The method of claim 12, wherein the target RNA embedding and the hit embedding each comprise graph-based embeddings and language-based embeddings.

14. The method of claim 12, further comprising performing an assay using a potential hit of the potential hits, wherein the binding model is updated based on the assay.

15. The method of claim 10, further comprising tuning the embedding model based on a binding success of the potential hits.

16. The method of claim 10, wherein determining the similarity score between the hit and each potential hit comprises computing a distance between the hit latent embedding and the potential hit latent embedding in an embedding space.

17. The method of claim 16, wherein the hit latent embedding is determined based on a SMILES string of the hit.

18. The method of claim 10, further comprising ranking the potential hits based on a molecular property of each of the potential hits.

19. The method of claim 18, wherein the molecule property comprises at least one of a synthetic accessibility score, a binding probability, or a 3D structure.

20. The method of claim 19, wherein the 3D structure is predicted using a diffusion model.
